(19) United States
(12) Patent Application Publication
(10) Pub. No.: US 2021/0401260 A1
(43) Pub. Date: Dec. 30, 2021

(54) SHOE WASHING AND STERILIZING DRYER

(71) Applicant: SMARTREUMBANGE INC, Gyeongsan-si, Gyeongsangbuk-do (KR)

(72) Inventor: Hae Jin YOUN, Gyeongsan-si, Gyeongsangbuk-do (KR)

(73) Assignee: SMARTREUMBANGE INC, Gyeongsan-si, Gyeongsangbuk-do (KR)

(21) Appl. No.: 16/616,439

(22) PCT Filed: Aug. 12, 2019
(86) PCT No.: PCT/KR2019/010197

§ 371 Date: Nov. 22, 2019

(30) Foreign Application Priority Data
Nov. 6, 2018 (KR) 10-2018-0135205

(51) Int. Cl. A47L 23/02; A47L 23/18; A47L 23/20; A46B 13/00; A46B 13/02; A61L 2/06

(52) U.S. Cl. CPC A47L 23/02; A47L 23/18; A47L 23/205; A61L 2202/17; A46B 13/02; A61L 2/06; A46B 2200/306; A46B 13/005

(57) ABSTRACT

The present invention relates to a shoe washing and sterilizing dryer for automatically washing, drying and sterilizing shoes such as sneakers, and so on, and more particularly, to a shoe washing and sterilizing dryer that is capable of allowing injection nozzles to spray a washing liquid to the form of fine particles into the surfaces of shoes and then allowing brushes to be rotatably moved up and down to perfectly wash the surfaces of the shoes, and also capable of allowing hot air generated from a heater to blow into the shoes and simultaneously allowing ultraviolet light to be irradiated onto the interiors of the shoes from UV lamps to perform both of drying and sterilizing processes for the shoes, thereby enhancing wearing comfort of the shoes and protecting a wearer from athlete's foot or various diseases.

### BACKGROUND OF THE INVENTION

### Field of the Invention

The present invention relates to a shoe washing and sterilizing dryer for automatically washing, drying and sterilizing shoes such as sneakers, and so on, and more particularly, to a shoe washing and sterilizing dryer that is capable of allowing injection nozzles to spray a washing liquid to the form of fine particles into the surfaces of shoes and then allowing brushes to be rotatably moved up and down to perfectly wash the surfaces of the shoes, and also capable of allowing hot air generated from a heater to blow into the shoes and simultaneously allowing ultraviolet light to be irradiated onto the interiors of the shoes from UV lamps to perform both of drying and sterilizing processes for the shoes, thereby enhancing wearing comfort of the shoes and protecting a wearer from athlete's foot or various diseases.

### Background of the Related Art

Generally, laundry like clothes can be easily laundered by a washing machine, but if shoes like sneakers are laundered by the washing machine, they may be much damaged. If the shoes are washed by using a laundry brush at home, further, it is very hard to do the laundry, so that recently, they are frequently taken to a laundry only for shoes.

The shoes become easily dirty because of soil or foreign matters attached to outer surfaces thereof, foreign matters entering interiors thereof through inlets, or a wearer's socks, and if the wearer walks or exercises, in addition, the shoes become contaminated well by his or her sweat so that they are in a bad sanitary state. However, it is difficult to wash or sterilize the shoes due to characteristics of the shoes.

Accordingly, a conventional representative technology wherein shoes are washed and sterilized at home is disclosed in Korean Patent No. 10-1524474 (entitled “shoe interior washing and sterilizing device for household”).

According to the conventional technology, the shoe interior washing and sterilizing device includes a body insertable into shoes and having a blower and a brush disposed on upper and lower portions of the front surface thereof and a sucking part disposed on the center of the front surface thereof, an operating pipe disposed integrally on the center of the rear end of the body and having an oscillator; and a fluctuating and coupling part disposed at the rear end portion of the operating pipe in such a manner as to be connected fluctuately with a connection pipe.

Under the configuration of the conventional technology, a sterilizing liquid is injectedly blown, sterilization is performed by vibrations of the washing brush, and foreign matters are separated from the shoes, at the front end portion of the body inserted into the shoes, and the foreign matters are collected to a water collection part through the connection pipe disposed at the rear end portion of the body. Accordingly, the conventional technology is simple in configuration so that it can keep the interiors of the shoes cleanly and sanitarily, while being small in size.

Further, a conventional representative technology wherein shoes are washed directly by a user is disclosed in Korean Patent No. 10-1564218 (entitled “shoe washing machine”).

According to the conventional technology, the shoe washing machine includes a water tank open at a portion of a top surface thereof and storing a washing liquid therein, a driving shaft disposed as the center of bottom of the water tank, a perforated plate fitted to one end outer periphery of the driving shaft to apply a rotary force to the driving shaft and having a plurality of through holes formed thereon, a fixing shaft disposed on the bottom of the water tank, an intermediate brush part having an intermediate brush coupled to one end of the fixing shaft in such a manner as to protrude upward from the center of the perforated plate, a driven shaft rotating together with rotation of the driving shaft, at least one or more driven brush parts each having a driven brush coupled to one end of the driven shaft in such a manner as to be exposed partially to the open portion of the water tank, and a power transmission unit transferring the rotary force of the driving shaft to the driven brush parts.

Under the configuration of the conventional technology, the perforated plate of the shoe washing machine is rotated by the user to wash the shoes, and accordingly, no separate power is used. Further, a driving device like a motor is not needed in the shoe washing machine, thereby advantageously preventing occurrence of electric shock accidents caused by electricity and reducing a manufacturing cost of a product.

According to the conventional technologies, the interiors of the shoes are washed and sterilized, and the shoes are washed by the perforated plate rotated directly by the user. In this case, however, the outer surfaces of the shoes are not washed perfectly by mean of the washing liquid, or after the shoes are washed, the interiors of the shoes cannot be dried and sterilized, thereby making wearing comfort of the shoes deteriorated and failing to protect a wearer from athlete's foot or various diseases.

### SUMMARY OF THE INVENTION

Accordingly, the present invention has been made in view of the above-mentioned problems occurring in the related art, and it is an object of the present invention to provide a shoe washing and sterilizing dryer that is capable of allowing injection nozzles to spray a washing liquid to the form of fine particles to the surfaces of shoes and then allowing brushes to be rotatably moved up and down to perfectly wash the surfaces of the shoes, and also capable of allowing hot air generated from a heater to blow into the shoes and simultaneously allowing ultraviolet light to be irradiated onto the interiors of the shoes from UV lamps.

It is another object of the present invention to provide a shoe washing and sterilizing dryer that is capable of allowing brushes to be rotatably moved up and down to perfectly wash the surfaces of the shoes, allowing hot air generated from a heater to blow into the shoes to completely dry the interiors of the shoes, and allowing ultraviolet light to be irradiated to the interiors of the shoes from UV lamps to sterilize the interiors of the shoes, thereby enhancing wearing comfort of the shoes and protecting a wearer from athlete's foot or various diseases.

To accomplish the above-mentioned objects, according to the present invention, there is provided a shoe washing and sterilizing dryer including: a housing configured to allow outer surfaces thereof to be surrounded with an external casing having an openable and closable cover and having a water collection container disposed on the underside thereof; a shoe mounting part disposed inside the housing to fit shoes thereto; first brushes disposed inside the housing in such a manner as to rotate by means of operations of first motors; second brushes disposed inside the housing in such a manner as to rotate by means of operations of second motors; moving units for moving up and down the first brushes and the second brushes; a washing liquid tank disposed on an upper portion of the rear side of the housing to store a washing liquid therein and having a pump adapted to pump the washing liquid; and a heater for blowing hot air into the shoe mounting part.

According to the present invention, desirably, the first brushes are attached to a fixing rod disposed horizontally in both side directions of the housing inside the housing, together with the first motors, and the fixing rod is attached to a frame disposed horizontally in both side directions of the housing at the front side of the interior of the housing.

According to the present invention, desirably, the second brushes are attached to top ends of upward inclined arms, together with the second motors, and undersides of the arms are attached to both sides of the frame disposed in both side directions of the housing inside the housing.

### BRIEF DESCRIPTION OF THE DRAWINGS

The above and other objects, features and advantages of the present invention will be apparent from the following detailed description of the embodiments of the invention in conjunction with the accompanying drawings, in which:

FIG. 1 is a side sectional view showing an installation state of a shoe washing and sterilizing dryer according to the present invention;

FIG. 2 is a perspective view showing an interior of the shoe washing and sterilizing dryer according to the present invention;

FIG. 3 is a front view showing the shoe washing and sterilizing dryer according to the present invention;

FIG. 4 is a side view showing the shoe washing and sterilizing dryer according to the present invention;

FIG. 5 is a side view showing an operating state of the shoe washing and sterilizing dryer according to the present invention;

FIG. 6 is a side view showing another operating state of the shoe washing and sterilizing dryer according to the present invention;

FIG. 7 is a side view showing yet another operating state of the shoe washing and sterilizing dryer according to the present invention; and

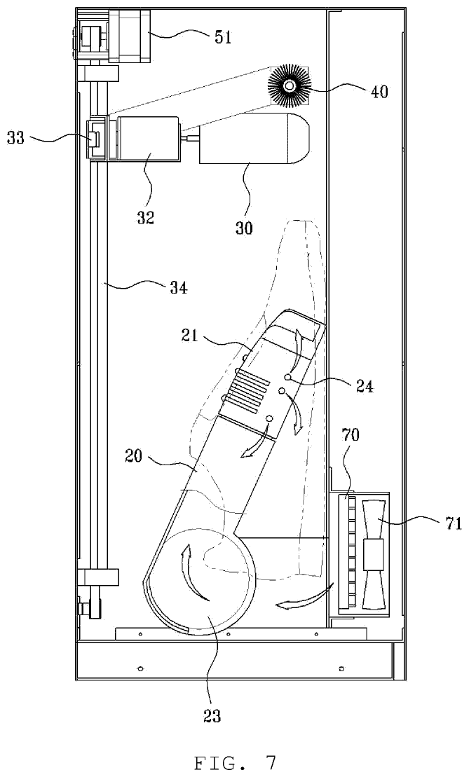

FIG. 8 is a side view showing another example of a second brush of the shoe washing and sterilizing dryer according to the present invention.

### DETAILED DESCRIPTION OF THE EMBODIMENTS

Hereinafter, the present invention is disclosed with reference to the attached drawings.

As shown, a shoe washing and sterilizing dryer according to the present invention includes: a housing 10 configured to allow outer surfaces thereof to be surrounded with an external casing 100 having an openable and closable cover 200 and having a water collection container 11 disposed on the underside thereof; a shoe mounting part 20 disposed inside the housing 10 to fit shoes thereto; first brushes 30 disposed inside the housing 10 in such a manner as to rotate by means of operations of motors 32; second brushes 40 disposed inside the housing 10 in such a manner as to rotate by means of operations of motors 40a; moving units 50 for moving up and down the first brushes 30 and the second brushes 40; a washing liquid tank 60 disposed on an upper portion of the rear side of the housing 10 to store a washing liquid therein and having a pump 61 adapted to pump the washing liquid; and a heater 70 for blowing hot air into the shoe mounting part 20.

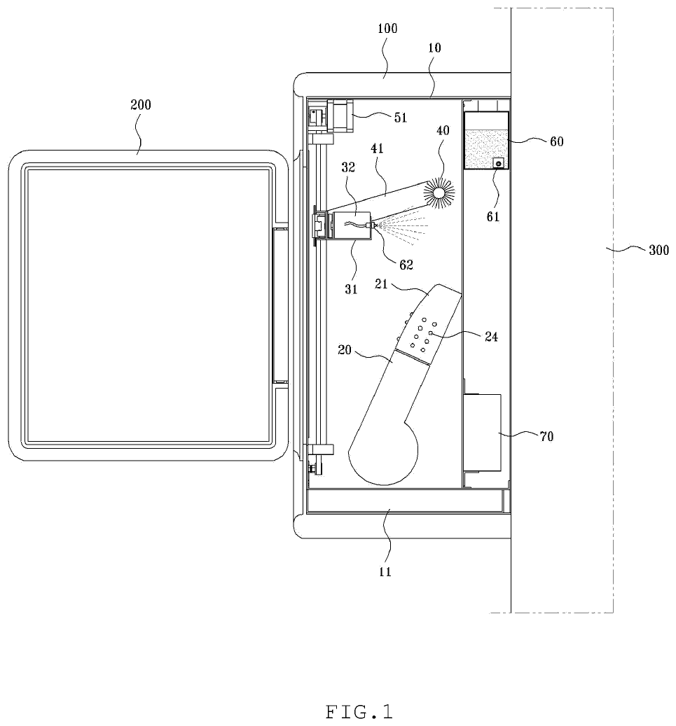

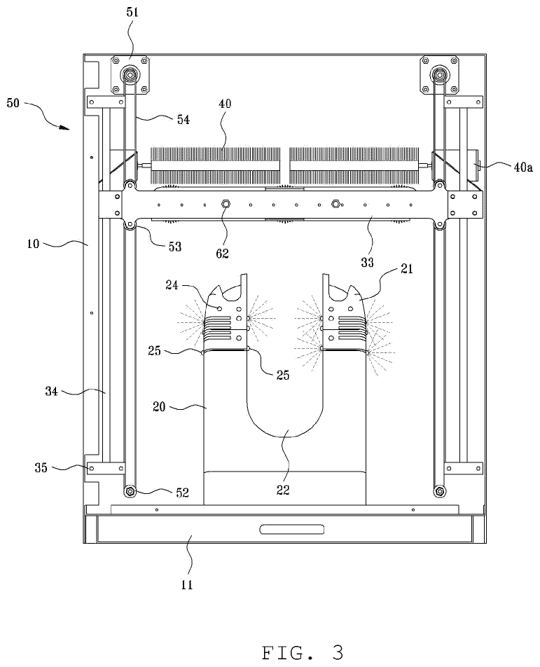

The housing 10 is a structure for forming an outer shape of the shoe washing and sterilizing dryer according to the present invention, and as shown in FIGS. 1 to 4, the housing 10 has a shape of a rectangular parallelepiped built with a plurality of panels in such a manner as to allow the outer surfaces thereof to be surrounded with the external casing 100 having the openable and closable cover 200. The cover 200 is openably and closably mounted on the front side of the external casing 100, and the external casing 100 has a control unit (LCD display window) adapted to control operations of the shoe washing and sterilizing dryer according to the present invention.

The housing 10 has a plurality of drain holes 12 penetrated into the bottom thereof and the water collection container 11 disposed on the underside thereof to store the waste washing liquid and foreign matters therein, and the water collection container 11 has a type of a drawer so that it can be drawnably fitted to the housing 10.

The housing 10 can be built on a floor in a house, and otherwise, it can be fixed as a wall-hanger type to an indoor wall 300. Accordingly, the cover 200 disposed on the front side of the external casing 100 is open to mount the shoes inside the housing 10, so that the shoes are washed, dried and sterilized.

The shoe mounting part 20 serves to fit the shoes thereto so that the shoes can be located inside the housing 10, and as shown in FIGS. 1 to 4, the shoe mounting part 20 is inclined upward from the internal bottom of the housing 10 and has holders 21 extended upward from both sides thereof to fit the shoes thereto and a long hole 22 formed between the holders 21,

Further, the shoe mounting part 20 has an injection hole 23 penetrated into a lower portion of the rear side thereof to allow hot air of the heater 70 to blow into an internal space thereof and a plurality of blowing holes 24 and a plurality of UV lamps 25 formed on the outer peripheries of the holders 21.

Through the plurality of blowing holes 24 formed on the outer peripheries of the holders 21, the hot air of the heater 70 blows into internal spaces of the shoes, and also, through the plurality of UV lamps 25 disposed on the outer peripheries of the holders 21, ultraviolet light is irradiated onto the interiors of the shoes.

Accordingly, the shoe mounting part 20 is configured to allow the shoes to be fitted to the holders 21, to allow the shoes to be dried in such a manner as to blow the hot air of the heater 70 into the internal spaces of the shoes through the injection hole 23 and the plurality of blowing holes 24, and to allow the shoes to be sterilized in such a manner as to irradiate the ultraviolet light onto the interiors of the shoes through the plurality of UV lamps 25.

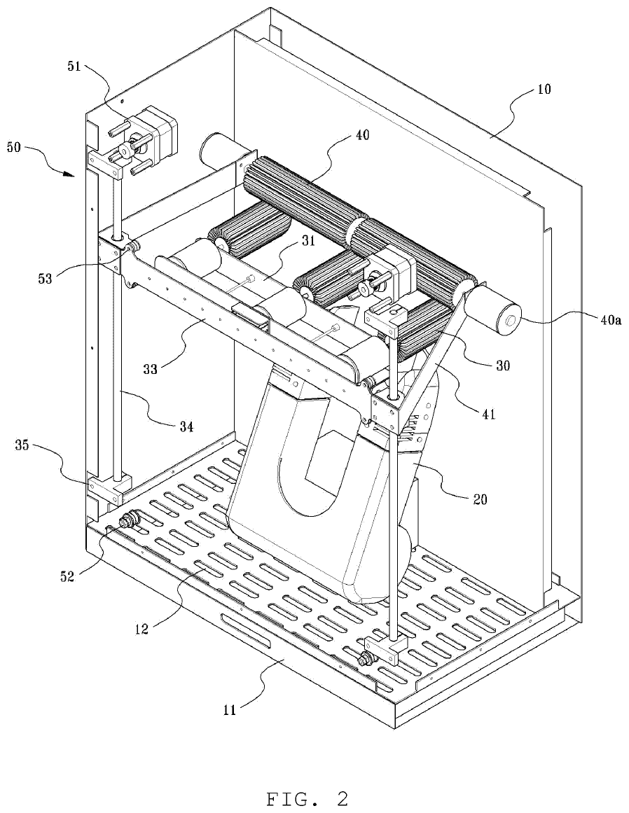

The first brushes 30 serve to wash both side surfaces of the shoes, and as shown in FIGS. 2 to 4, they are disposed inside the housing 10 in such a manner as to be arranged horizontally. As shown, the number of first brushes 30 is three.

The first brushes 30 are attached to a fixing rod 31 disposed horizontally in both side directions of the housing 10 inside the housing 10, together with the motors 32, and the fixing rod 31 is attached to a frame 33 disposed horizontally in both side directions of the housing 10 at the front side of the interior of the housing 10.

The frame 33 whose both sides are fitted to guide bars 34 disposed vertically at both sides of the interior of the housing 10 in such a manner as to be movable up and down, and tops and undersides of the guide bars 34 are fixed to both sides of the interior of the housing 10 by means of brackets 35.

Accordingly, the first brushes 30 rotate by means of operations of the motors 32 to remove soil or foreign matters out of both side surfaces of the shoes, and in this case, the first brush 30 located in the middle of the first brushes 30 is placed inside the long hole 22 of the shoe mounting part 20 at the time when it is moved up and down by means of the moving units 50 as will be discussed later.

The second brushes 40 serve to wash the top surfaces of the shoes, and as shown in FIGS. 1 to 4, the second brushes 40 are disposed horizontally in left and right directions at both sides of the interior of the housing 10 above the first brushes 30.

The second brushes 40 are attached to top ends of upward inclined arms 41, together with the motors 40a, and in this case, the second brushes 40 are facingly located inside the arms 41, while the motors 40a being located outside the arms 41. Also, the undersides of the arms 41 are attached to both sides of the frame 33 disposed in both side directions of the housing 10 inside the housing 10.

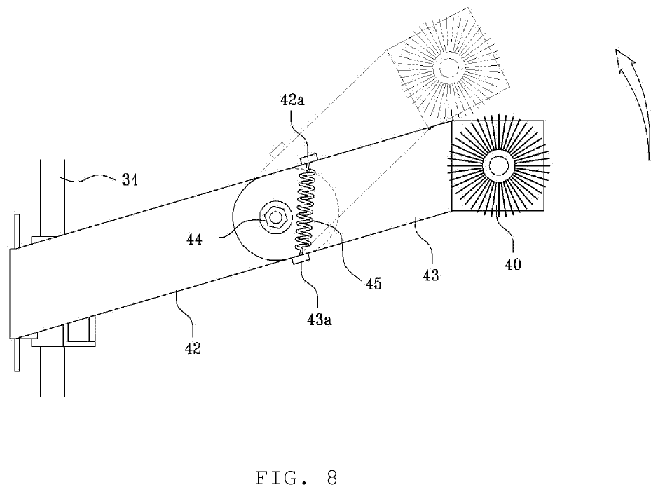

Referring to FIG. 8 showing another example of the second brushes 40, on the other hand, each second brush 40 is attached to a first arm 42 and a second arm 43 divided and rotated by means of a hinge 44, and fixing members 42a and 43a are mounted on the first arm 42 and the second arm 43 in such a manner as be connected to a spring 45.

In such a configuration, if external forces are applied to the second brushes 40 according to the shapes of the shoes in a process where the second brushes 40 rotate by means of operations of the motors 40a to wash the top surfaces of the shoes, the second brushes 40 rotate upward around the hinges 44 to wash the shoes, and if the washing is finished to remove the second brushes 40 out of the shoes, the second arms 43 rotate downward by means of the elasticity of the springs 45 to allow the second brushes 40 to be returned to their original state.

Accordingly, the second brushes 40 rotate by means of the operations of the motors 40a to remove soil or foreign matters out of the top surfaces of the shoes, and if external forces are applied to the second brushes 40 according to the shapes of the shoes, the second brushes 40 rotate upward around the hinges 44 to wash the shoes.

The moving units 50 serve to move up and down the first brushes 30 and the second brushes 40 to wash the shoes, and as shown in FIGS. 2 to 4, each moving unit 50 includes a motor 51, a rotating roller 52, a fixing roller 53, and a belt 54.

The moving units 50 are configured to have the motors 51 fixed to both sides of the upper side of the housing 10, to have the rotating rollers 52 disposed on both sides of the lower side of the housing 10, to have the fixing rollers 53 disposed on upper and lower portions of both sides of the frame 33, and to have the belts 54 wound on driving pulleys of the motors 51, the rotating rollers 52 and the fixing rollers 53.

A timing belt with teeth formed at the inner surface thereof is desirably used as each belt 54, and if the motors 51 are driven, the belts 54 wound on the driving pulleys and the rotating rollers 52 are moved up and down to allow the fixing rollers 53 to be moved up and down, so that the frame 33 is moved up and down to allow the first brushes 30 and the second brushes 40 to be moved up and down. At this time, the frame 33 is stably moved up and down along both side guide bars 34.

Accordingly, the moving units 50 are configured to allow the belts 54 to be moved up and down through the operations of the motors 51, so that the frame 33, the first brushes 30, and the second brushes 40 are moved up and down to wash the shoes.

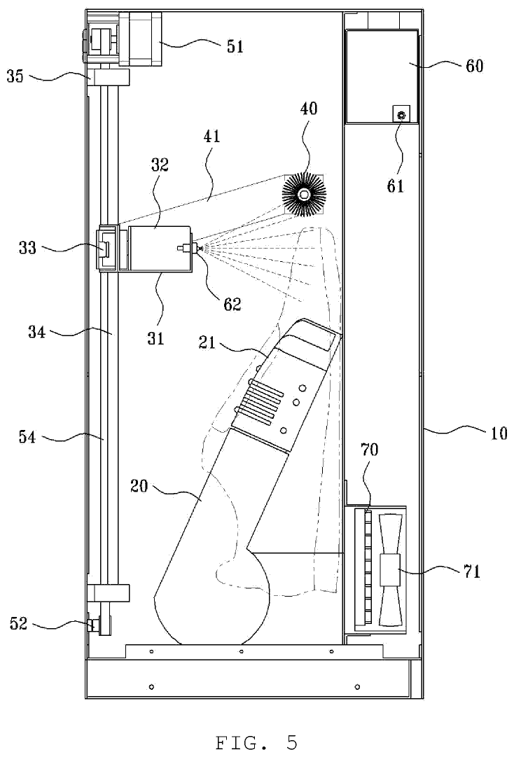

The washing liquid tank 60 serves to store the washing liquid therein, to inject the washing liquid, and to apply the washing liquid to the surfaces of the shoes, and as shown in FIGS. 1 and 5, the washing liquid tank 60 is disposed on the upper portion of the rear side of the housing 10 to store the washing liquid therein and has the pump 61 adapted to pump the washing liquid.

The washing liquid tank 60 desirably has a cover disposed on top thereof to allow the washing liquid to be put therein by a user and a plurality of injection nozzles 62 disposed on the fixing rod 31 to which the first brushes 30 are attached toward the rear side of the housing 10 in such a manner as to be connected to the pump 61 by means of a hose.

Accordingly, the washing liquid stored in the washing liquid tank 60 is pumped to the injection nozzles 62 by means of the pump 61 and is thus sprayed to the form of fine particles through the injection nozzles 62, so that it can be applied to the surfaces of the shoes fitted to the holders 21 of the shoe mounting part 20.

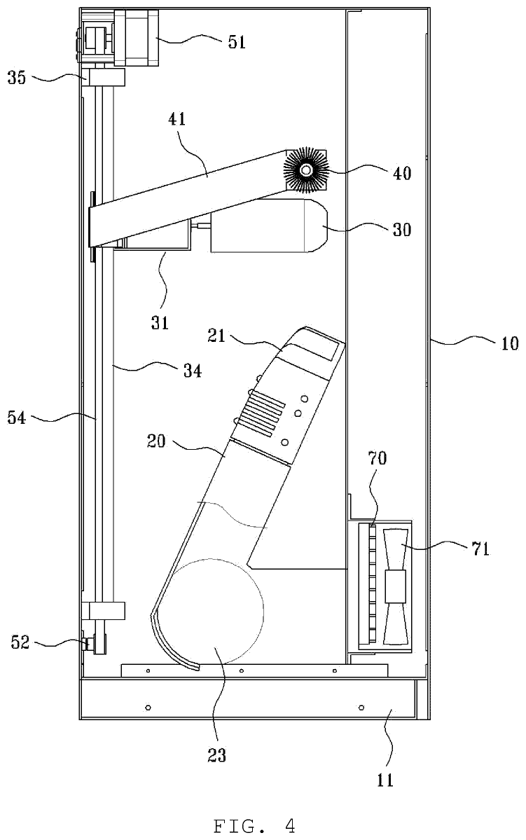

The heater 70 serves to blow hot air into the shoes to dry the shoes well, and as shown in FIGS. 4 to 6, the heater 70 is disposed on the lower portion of the rear side of the housing 10 and has a fan 71 adapted to blow hot air.

The heater 70 is disposed on the lower portion of the rear side of the housing 10, that is, on the rear side of the shoe mounting part 20 in such a manner as to communicate with the injection hole 23 of the shoe mounting part 20 and to also blow the hot air to the outside of the housing 10.

Accordingly, the heater 70 is configured to allow the hot air to blow through the fan 71, to allow the hot air to be supplied to the internal space of the shoe mounting part 20, and to allow the hot air to blow into the internal spaces of the shoes through the plurality of blowing holes 24 of the holders 21 of the shoe mounting part 20, thereby performing a drying process.

Under the above-mentioned configuration, an explanation on the operating procedure of the shoe washing and sterilizing dryer according to the present invention will be given.

First, the washing liquid is put into the washing liquid tank 60, and the cover 200 is open to fit the shoes to the holders 21 of the shoe mounting part 20. Next, the cover 200 is closed to allow washing, drying and sterilizing processes to be carried out through the control unit (LCD display window).

First, as shown in FIG. 5, the motors 51 of the moving units 50 are driven to move the injection nozzles 62 down, and thus, the injection nozzles 62 becomes close to the shoes. Next, the pump 61 is operated to spray the washing liquid to the form of fine particle through the injection nozzles 62, and thus, the washing liquid is applied to the surfaces of the shoes.

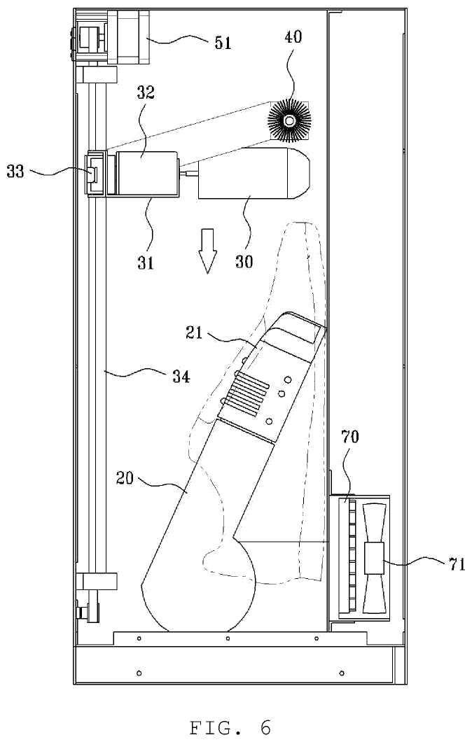

Next, as shown in FIG. 6, the first brushes 30 and the second brushes 40 rotate by means of the operations of the motors 32 and 40a, and simultaneously, the motors 51 of the moving units 50 are operated to move down the first brushes 30 and the second brushes 40, so that while the first brushes 30 and the second brushes 40 are removing the soil or foreign matters attached to both sides and top surface of the shoes, the washing process is carried out.

At this time, the soil or foreign matters removed from the shoes and the waste washing liquid are collected to the water collection container 11 through the drain holes 12 formed on the bottom of the housing 10, and if the washing is finished, the motors 51 of the moving units 50 are operated reversely to move up the first brushes 30 and the second brushes 40.

Next, the heater 70 and the fan 71 are operated to blow the hot air generated from the heater 70 into the shoes through the blowing holes 24 of the holders 21, thereby carrying out the drying process, and simultaneously, the UV lamps 25 irradiate ultraviolet light onto the interiors of the shoes, thereby carrying out the sterilization process. Accordingly, the washing, drying and sterilization for the shoes are all finished.

As described above, the shoe washing and sterilizing dryer according to the present invention is configured to allow the injection nozzles to spray the washing liquid to the form of fine particles to the surfaces of the shoes and to then allow the brushes to be rotatably moved up and down to perfectly wash the surfaces of the shoes, and also configured to allow hot air generated from the heater to blow into the shoes and simultaneously to allow the ultraviolet light to be irradiated onto the interiors of the shoes from the UV lamps to perform both of drying and sterilizing processes for the shoes, thereby enhancing wearing comfort of the shoes and protecting a wearer from athlete's foot or various diseases.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A shoe washing and sterilizing dryer comprising:
a housing configured to allow outer surfaces thereof to be surrounded with an external casing having an openable and closable cover and having a water collection container disposed on the underside thereof;
a shoe mounting part disposed inside the housing to fit shoes thereto;
first brushes disposed inside the housing in such a manner as to rotate by means of operations of motors;
second brushes disposed inside the housing in such a manner as to rotate by means of operations of motors;
moving units for moving up and down the first brushes and the second brushes;
a washing liquid tank disposed on an upper portion of the rear side of the housing to store a washing liquid therein and having a pump adapted to pump the washing liquid; and
a heater for blowing hot air into the shoe mounting part.

2. The shoe washing and sterilizing dryer according to claim 1, wherein the first brushes are attached to a fixing rod disposed horizontally in both side directions of the housing inside the housing, together with the motors, and the fixing rod is attached to a frame disposed horizontally in both side directions of the housing at the front side of the interior of the housing.

3. The shoe washing and sterilizing dryer according to claim 1, wherein the second brushes are attached to top ends of upward inclined arms, together with the motors, and undersides of the arms are attached to both sides of the frame disposed in both side directions of the housing inside the housing.
